(19) United States
(12) Patent Application Publication
(10) Pub. No.: US 2006/0154253 A1
(43) Pub. Date: Jul. 13, 2006

(54) Method for the validated construction of arrays

(76) Inventors: Ramon Guimil, Heidelberg (DE); Matthias Scheffler, Leutershausen (DE); Barbro Beijer, Nussloch (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/540,517

(22) PCT Filed: Dec. 23, 2003
(86) PCT No.: PCT/EP03/14826

§ 371 Date: Feb. 9, 2006

(30) Foreign Application Priority Data
Dec. 23, 2002 (DE) 102 60 592.0

(51) Int. Cl. C12Q 1/68; C07F 3/08; C07D 311/88; C07D 311/02

(52) U.S. Cl. 435/6; 435/287.2; 549/227; 549/286; 556/119; 977/924

(57) ABSTRACT

The present invention relates to a method for validating the synthesis of arrays, in particular of biopolymers, by step-by-step construction from protected and labeled synthesis building blocks.

The present invention relates to a method for validating the synthesis of arrays, in particular of biopolymers, by step-by-step construction from protected and labeled synthesis building blocks.

According to the current prior art, the receptors (probes) of an array are immobilized by covalent or noncovalent interaction or synthesized in situ on the solid phase. The building blocks of a chemical polymerization process are generally referred to as “synthons”. The functional groups of a synthon allow a targeted chemical reaction with suitable other functional groups on a second synthon. These reactive centers are usually masked by “protective groups” which may be specifically removed in a suitable chemical environment, thereby enabling the synthesis process to be controlled, since only those functional groups which do not carry any protective group are reacted. Depending on the particular synthesis strategy, protective groups may generally be removed by altering chemical or physicochemical environmental parameters such as the redox potential, the pH or the temperature, but also by introducing electromagnetic energy, for example via irradiation with light of a particular wavelength.

The sequential polymerization by way of condensation to a solid phase, introduced by Merryfield, has proved its worth for sequence-specific synthesis of oligomers such as oligonucleotides, oligonucleotide derivatives, peptides or carbohydrates, which are constructed from different, but a finite number of, monomer units (R. B. Merryfield (1963), J. Am. Chem. Soc. 85: 2149-2154; R. B. Merryfield (1965) Science 150: 179-185). The synthesis according to Merryfield initiates at a support-bond functional group which may carry a protective group and which can be activated by removal of the latter. This permits coupling of a next monomer supplied in solution, which itself, after polymerization via condensation, is available as starting point for a further polymerization step. Successive repetition of activation and subsequent polymerization via condensation finally results in the synthesis of the desired oligomer.

The products of a conventional solid phase-supported (bio)polymer synthesis are removed from the solid phase by cleavage of a predetermined breaking point, for example by hydrolysis of an oxalate or succinate, and thus become accessible to the known analytical methods such as NMR, HPLC, CE, MS, phosphoimager, etc. By combining a cleavable spacer amidite (predetermined breaking point=esters, sulfones, etc.) and a fluorescent branched phosphoamidite, it is possible to transfer the above-described strategy in accordance with a construction kit approach also to arrays. The cleavage products are 3′-fluorescently labeled. In this way, it is possible to draw conclusions about the coupling efficiency (U.S. Pat. No. 6,238,862 B1, J. Burmeister, A. Azzawi, G. v. Kiedrowksi, Tetrahedron Lett. 1995, 3667-68]. The same result is achieved when using a trifunctional molecule which provides one functionality for polymer synthesis, one for labeling and one for anchoring on the solid phase. The third functionality in addition also still has a labile bond [U.S. Pat. No. 5,290,925].

A further possible quality control is to fuse additional cleavable phosphate group-labeled phosphoamidites to the 5′ ends. The reporter group signal is then used for controlling the quality of the synthesis. 5′-phosphorylated probes are obtained after removing the reporter group [Beier, WO 00/15837].

The method for producing finished chemical surfaces which is described in WO 01/36086 has the object to introduce functional groups in situ, i.e. during the functionalization process and to provide a surface of specific chemical or physicochemical properties, adapted to the later application process

Other methods use “starburst” dendrimers in order to increase the number of reaction sites, thereby ultimately achieving an increase in the density of functional groups on the surface and indirectly in an improvement of the detection signal (M. Beier, J. D. Hoheisel (1999) Nucleic Acids Res. 27: 970-1977) or better signal-to-noise ratios (Niemeyer et al. (2000), Chembiochem. 2:685-694).

The use of so-called “inverted synthons” which are nucleosides whose 5′ hydroxy functions carry a phosphoamidite group and whose 3′ hydroxy functions carry a protective group, for example DMT or Nppoc, enables the chemical oligonucleotide synthesis to be carried out in the natural 5′→3′ direction. The altered orientation on the array surface opens up a further field of application for DNA/RNA microarrays, enabling, inter alia, the enzymic chain extension by polymerases [WO 00/61594; WO 01/55451; M. C. Pirrung, Org. Lett. 2001, 3,8, 1105-1108].

Site-specific deprotection of individual protective groups is absolutely necessary for an in situ array synthesis. One possible space-resolved synthesis makes use of the process of photolithography and will now be more particularly described using the synthesis of DNA as an example: the light-controlled synthesis of nucleic acid chips uses photolabile nucleoside derivatives. The chain of nucleic acid fragments is constructed here usually by means of phosphoamidite synthons. The building blocks carry in each case a temporary photoprotective group which may be removed by incident light. The principle of the synthesis provides for a cyclic sequence of condensation and deprotection steps (by light). The efficiency with which such a light-controlled synthesis can take place is determined essentially by the photolabile protective groups used, in particular by the efficiency with which they can be removed in the irradiation step.

The photoprotective groups used to date for light-controlled synthesis are normally the protective groups NVOC (S. P. A. Fodor et al., Science 251 (1991), 767 ff.), MeNPOC (A. C. Pease et al., Proc. Natl. Acad. Sci. 91 (1994), 5022 ff.), DMBOC (M. C. Pirrung, J. Chem. 60 (1995), 1116 ff.) and NPPOC (A. Hassan et al., Tetrahedron 53 (1997), 4247 ff.). Further known photolabile protective groups in nucleoside and nucleotide chemistry are o-nitrobenzyl groups and their derivatives (cf. e.g. Pillai, Org. Photochem. 9 (1987), 225; Walker et al., J. Am. Chem. Soc. 110 (1988), 7170). Further photolabile protective groups which have been proposed are the 2-(o-nitrophenyl)ethyl group (Pfleiderer et al., In: “Biophosphates and their Analogs—Synthesis, Structure, Metabolism and Activity”, ELSEVIER Science Publishers B. V. Amsterdam (1987), 133 ff.) and derivatives thereof (WO 97/4434, WO 96/18634, WO 02/20150).

The photolabile protective groups currently used for light-controlled synthesis of nucleic acids (e.g. NVOC, MeNPOC, NPPOC) are generally distinguished by a comparatively low absorption coefficient at the wavelength of the incident light. Irradiation of photolabile nucleoside derivatives normally takes place using high pressure Hg lamps at a wavelength of 365 nm. The result of the low absorption coefficient of the photolabile protective group used at this wavelength is that only a very small proportion of the incident light can be utilized for excitation of the molecules. In addition, the photolabile protective groups used are mostly colorless derivatives. The result of this in turn is that it is not possible during the synthesis to detect by simple spectroscopic methods whether the photolabile protective group is still present on the nucleoside derivative or has already been partly or completely abstracted by the input of light. The abstraction process can thus be followed only with difficulty, if at all.

A development of the photolithographic process is the use of fluorescent photolabile protective groups. In the case of benzylic photolabile protective groups, molecules of the type ArCR₁R₂—OC(O)X are used, where Ar may be a fused aromate, for example pyrene, and R₁R₂ may be substituted aromates. The fluorescence of the Pymoc group is used in order to estimate the functionalization density [WO 98/39348]. Another procedure is the labeling of nitrobenzyl derivatives [C. Muller et al, Helv. Chim Acta 2001, 84, 3735-3740]. The fluorescence signals of coumarin can be used for quality control.

An alternative to photolithography is DNA synthesis based on tandem protective groups. This type of protective group is obtained by combining two protective groups established in nucleotide chemistry, for example a photolabile Nppoc-(2-[2-nitrophenyl]-propyloxycarbonyl chloride) group and an acid-labile dimethoxytrityl group, and the derivatives deriving therefrom (DE 101 32 025.6, DE 102 60 591.2 and PCT EP 02/07389). Removal of the colored trityl cation of the two-stage protective groups, which cation possesses a substantially higher absorption coefficient than the cleavage products of other photodeprotection processes based on benzyl or nitrobenzyl, furthermore opens up the possibility of direct online process control. This results in an improved quality control for biochips.

Efforts to influence the properties of the trityl protective group have a long tradition [A. Taunton-Rigby et al., J. Org. Chem. 1972, 37, 956-964]. Work by some groups has increased the hydrophobic character for purification on the basis of RP chromatography [R. Ramage et al. Tetrahedron Lett. 34 (1993) 7133; H. Seliger et al., Angew. Chem. 1981, 93, 709], others have varied the exocyclic groups so as to reverse the cleavage conditions (base-labile trityls) [M. Sekine et al., Bull. Chem. Soc. Jpn. 1985, 58, 336; M. Sekine et al., J. Am. Soc. 1986, 108, 4581] or to be able to carry out the cleavage under particularly gentle conditions, for example hydrazinolysis of 4-(9-fluorenenylmethoxyoxycarbonyl)oxy- or amino-4′,4″-dimethoxytriyl [E. Happ et al., Nucleosides and Nucleotides 1988, 7, 813-816]. The fluorescently labeled trityl compounds strictly serve as protective group with unusual lability properties.

It is furthermore possible to lower the detection limits of the trityl-containing nucleotides and oligonucleotides in HPLC or TLC by incorporating pyrene into the trityl backbone. This compound corresponds to the type: P^{m}-C*, where P^{m} is protective group P which may carry a label m, for example a fluorescent dye, and C* is a functional group to be protected which corresponds to the 5′ hydroxy group of a nucleoside [J. L. Fourrey et al. Tetrahedron Lett. 1987, 28, 5157].

U.S. Pat. No. 5,410,068 describes a similar approach in order to reversibly modify biological compounds for identification, separation and purification. Fluorescently labeled trityl groups of the type M-L-P-C* are described. M is a label which is used for identifying the molecule, L is a spacer, P is a trityl protective group and C* is the functional group of a biomolecule.

The array-supported polymer synthesis of partly complex substance libraries usually faces the problem that whether the synthesis has been successful is found out only after said synthesis has been carried out. This is further complicated by the fact that the surfaces of the arrays generated in situ may sometimes be difficult to access. This can considerably impair the quality control by standard methods of analyzing surfaces. It would therefore be desirable, for economic reasons, to possess a method which makes it possible to obtain information about the quality early, during processing.

It was an object of the present invention to develop a method for preparing solid phase-bound arrays, which allows improved quality control compared with the methods of the prior art.

This object is achieved by a method for preparing a coated support, comprising the following steps:
(a) providing a support whose surface has reactive groups,
(b) constructing a functionalized surface on said support by synthesizing step-by-step a receptor comprising a linker element and a probe element made of synthon building blocks,
   characterized in that the synthesis process is monitored (i) by introducing one or more synthon building blocks with at least one detectable labeling group into the linker element and (ii) by introducing one or more synthon building blocks with at least one detectable labeling group into the probe element of the receptor.

The present invention relates to a method in which the quality of the array surface is checked but, preferably, not influenced and which enables one or more or all of the subsequent steps of a biopolymer synthesis to be monitored online, i.e. in order to increase efficiency, it is possible to check one or more steps, for example the first, the n-th, the 2n-th, second last or/and last etc., step, in the synthesis of linkers and probes.

The synthesis process of the invention comprises introducing one or more synthon building blocks having at least one detectable labeling group into the linker element and introducing one or more synthon building blocks having at least one detectable labeling group into the probe element of the receptor. In this connection, it is possible to use, for example when constructing the linker element or/and the probe element, in each case a single labeled synthon building block or a plurality of labeled synthon building blocks, for example two, three, four, etc., labeled synthon building blocks. All synthon building blocks for synthesizing the linker element or/and the probe element may, optionally, comprise at least one detectable labeling group. In a preferred embodiment of the method of the invention, the first and the third step of constructing the linker element or/and the probe element are carried out using a labeled synthon building block. Steps without the use of labeled synthon building blocks may be carried out in the conventional manner.

Aside from the functionalization density which directly correlates to the later probe density, a uniform distribution of the functional groups is of crucial importance. In order to assess the homogeneity, a multiplicity of labels can be integrated into the syntheses of the biopolymers such as, for example, nucleic acids such as DNA or RNA, nucleic aid analogs such as PNA or LNA, oligonucleotides (independently of the direction of synthesis), saccharides, carbohydrates, peptides, proteins and further mixed forms or else derivatives of combinatorial chemistry. The biopolymers may be synthesized in any direction, for example in the 5′→3′ or/and in the 3′→5′ direction for nucleic acids. Accordingly, optical methods such as absorption, emission/light diffraction, light scattering or ellipsometry, or else other methods such as radioactivity, plasmon resonance or electronic methods such as electron diffraction or electrical signals etc., may be employed for analysis. Particular preference is given here to the analytical methods which can be integrated into a system for synthesizing arrays or biochip supports according to WO 00/13018.

It is possible to use in the method of the invention synthon building blocks comprising a plurality of labeling groups detectable independently. Thus it is possible to use labeling groups for introduction into the linker element which are detectable beside the labeling groups for introduction into the probe element.

The synthesis of the linker element and the synthesis of the probe element according to the present invention comprise preferably in each case a plurality of steps, the particular elements being constructed from a plurality of synthon building blocks. The linker element is constructed from one or more nonfunctional synthon building blocks which differ from the functional synthon building blocks for the probe element. Preferred examples of linker synthons are alkyl radicals, oligoethylene glycol radicals or combinations of alkyl and aryl radicals.

The linker molecules synthesized on the support comprise functional groups which permit the coupling of further linker-synthon building blocks or, after the linker synthesis has finished, probes or probe building blocks. Functional groups of the linker molecules may be selected, for example, from —OR, —NR₂, —SR, —PO₃R₂, —CN, —SCN, —COR′ and —OCOR′, where R is H or a protective group and R′ is H or a protective group or —OR, —NR₂ or SR. R and R′ may furthermore be alkyl, aryl, alkenyl and/or allyl radicals and/or further suitable organic radicals.

Linker synthesis is followed by the coupling of probes, which may likewise be carried out by step-by-step synthesis from synthesis building blocks, depending on the synthesis strategy used, for example peptide, oligonucleotide or carbohydrate synthesis on the solid phase, or by site-specific and/or non-site-specific immobilization of complete probes. Particularly preferred building blocks for oligonucleotide synthesis are phosphoamidites.

The support may in principle be selected randomly, for example from particles, in particular magnetic particles, microtiter plates and microfluidic supports (such as, for example, fluidic microprocessors) and may have a surface selected from glass, metals, semimetals, metal oxides or plastic. Particular preference is given to the microparticles disclosed in PCT/EP 99/06315 and the supports disclosed in PCT/EP 99/06316 and PCT/EP 99/06317, which have planar surfaces and surfaces provided with microchannels (cross section: e.g. 10-1000 μm), respectively. Explicit reference is made to the disclosure of the documents mentioned.

The receptor molecules may be synthesized on the entire surface of the support or else site-specifically at selected reaction sites.

Detectable labeling groups are used for at least one of the synthon building blocks for introduction into the linker element and for at least one of the synthon building blocks for introduction into the probe element. Optionally, all synthon building blocks for synthesis of the linker element and of the probe element may comprise at least one detectable labeling group.

Detectable labeling groups may be removed during or/and after synthesis of the linker element and of the probe element. Different labeling groups may be removed at different points in time of the process.

Preference is given to employing the following types of compounds in the method relevant to the invention. Surface control for incorporation into the linker element may employ in particular compounds of the following group I (types I-VII):

| | |
|---|---|
| Type I: | P^{m}-C* |
| Type II: | M-L-P^{m}-C* |
| Type III: | P^{m}L-V(L′-H-L″-M)-L″′-C* |
| Type IV: | P^{m}′P^{m}-L-V(L′-H-L″-M)-L″′-C* |
| Type V: | M-L-P^{m}′P^{m}-L-V(L′-H-L″-M)-L″′-C* |
| Type VI: | P^{m}′P^{m}-C* |
| Type VII: | M-L-P^{m}′P^{m}-C* |

P^{m} is a protective group P which may carry a label m for detection, with the labels M or/and m, optionally, being bound to P with a linker and said protective group and labels being compatible with synthesis chemistry.

P^{m}′ is a protective group orthogonal to P^{m}, which may carry a label m′ for detection. The protective group P^{m}′ may be selectively removed under conditions under which the protective group P^{m} is stable. P^{m}′ may be, for example, a protective group which can be removed photochemically by illumination and P^{m} may be a protective group which can be removed by chemical methods, for example treatment with acid or base.

M, m and m′ are labels which may be used for detection. M, m and, optionally, m′ are detectable independently. They may, optionally, also be removable independently, for example in the compounds of types III, IV or VI. Conveniently, the labels are also compatible with synthesis chemistry.

L to L″′ are any spacers, for example organic groups such as, for example, alkylene groups, which, optionally, may comprise heteroatoms such as O, N, P and S.

H is a cleavable group, for example an ester, disulfide, sulfone or diol group,

V is a trifunctional molecule/atom such as, for example, a nucleoside, trihydroxyalkyl or dihydroxyaminoalkyl.

C* is a functional group or a functionalized support surface, in particular a linker building block.

The compounds of types II to VII normally comprise a plurality of labeling groups, M, m or/and m′. However, those compounds which carry only one labeling group are also included, i.e., for example, the protective group P^{m} in the compounds need not comprise any labeling group, as long as said compound comprises at least one other labeling group, for example M.

Among the compounds of group I, particular preference is given to compounds of types I or/and II.

Probe control for incorporation into the probe element may employ in particular compounds of the following group II (types I, II and VI-X):

| | |
|---|---|
| Type I: | P^{m}-C* |
| Type II: | M-L-P^{m}-C* |
| Type VI: | P^{m}′P^{m}-C* |
| Type VII: | M-L-P^{m}′P^{m}-C* |
| Type VIII: | P^{m}-L-H-L′-C* |
| Type IX: | P^{m}′P^{m}-L-H-L′C* |
| Type X: | M-L-P^{m}′P^{m}-L-H-L″-C* |

P^{m} is a protective group (P) which may carry a label (m) for detection, with the label M (m), optionally, being bound to (P) via a linker and said protective group and said label being compatible with synthesis chemistry.

P^{m}′ is a protective group orthogonal to P^{m}, which may carry a label (m′) for detection. The protective group P^{m}′ may be selectively removed under conditions under which the protective group P^{m} is stable. P^{m} may be, for example, a protective group which can be removed photochemically by illumination and P^{m}′ may be a protective group which can be removed by chemical methods, for example treatment with acid or base.

M, m and m′ are labels which may be used for detection. M, m and, optionally, m′ are detectable independently. They may also be removable independently. Conveniently, the labels are also compatible with synthesis chemistry.

L to L″ are any spacers, for example organic groups such as, for example, alkylene groups, which, optionally, may comprise heteroatoms such as O, N, P and S.

H is a cleavable group, for example an ester, disulfide, sulfone or diol group,

C* is a probe building block, for example a nucleotide, a nucleotide analog, an amino acid, an amino acid analog etc.

The compounds of types II and VI to X comprise one or more labeling groups, i.e. the protective group P^{m} or/and P^{m} need not comprise any labeling group, as long as the compound comprises at least one other labeling group.

Among the compounds of group II, particular preference is given to types I or/and II.

In order to illustrate the method of the invention, the individual types will be illustrated by way of general examples of phosphoamidite chemistry and later by way of specific embodiments. The compounds described have been labeled in such a way that quality assessment is possible on the basis of the emission of their one to three fluorescent labels and, partially, on the basis of their absorption. The embodiments can, of course, also be transferred to other synthesis strategies or/and labeling groups.

A preferred embodiment is as follows: first, a labeled synthon is fused to the functionality of the support surface. After fluorescence has been measured, the labels are removed either at the next deprotection step, in accordance with a step-by-step biopolymer synthesis, or at the end of the synthesis so that they cannot distort the detection signal of the hybridization.

In the case of trityl-containing protective groups or of labeled photoprotective groups, a secondary analysis, the spectroscopic analysis of the colored trityl cations and, respectively, of the now colored (in comparison with the cleavage products of conventional photoprotective groups) and possibly fluorescent cleavage products of the photodeprotection process, comes in useful. The data obtained are compared with an evaluation criterion and, after a positive assessment, processed further.

The compound types, for example phosphoamidites III-V, are compounds which may make accessible several quality assurance methods at the same time: homogeneity check is carried out via label M or m which may be detectable independently of one another, for example two fluorescent dyes or fluorescence in combination with radiolabeling, with m and M which are part of the P^{m′}P^{m}, P^{m} and M-L-P^{m}′P^{m} groups being removed before the next coupling step. The labeling groups may, optionally, for example with compounds of types III and/or IV, also be removed at a later time, for example during the final deblocking step. Expediently, all labeling groups are removed before the final deblocking step, so that their signal cannot interact with the measured signal on the support.

If the starting groups of the P^{m′}P^{m}, P^{m} and M-L-P^{m}′P^{m} cleavage products which make spectroscopic online process control possible are incorporated into nucleoside phosphoamidites, then compounds are obtained which may be employed for probe quality control.

The following preferred examples of the individual types of compounds are suitable:

| | | |
|---|---|---|
| Type I: | P^{m}-C* | triplet-sensibilized Nppoc |
| Type II: | M-L-P^{m}-C* | fluorescently labeled |
| | | protective group (coumarin |
| | | Nppoc) |
| Type VI: | P^{m}′P^{m}-C* | fluorescently labeled tandem |
| | | group (pyrene Nppoc) |
| Type VII: | M-L-P^{m}′P^{m}-C | fluorescently labeled tandem |
| | | group (coumarin Nppoc) |
| Type VIII: | P^{m}-L-H-L′-C | (Nppoc or DMT succinate |
| | | derivatives) |
| Type IX: | P^{m}′P^{m}-L-H-L′C | fluorescently labeled tandem |
| | | group (pyrene Nppoc) succinate |
| Type X: | M-L-P′P^{m}-L-H-L″-C* | fluorescently labeled tandem |
| | | group |

The methods of conventional quality control for polymer syntheses (CE, HPLC, MS etc.) can essentially be transferred to the products of the array synthesis, taking into account the three-dimensional construction of arrays and in particular by using molecules of type VIII-X, provided that the functionalization process of the surface provides a sufficiently high functionalization density.

The invention further relates to compounds of the general structure (XI): 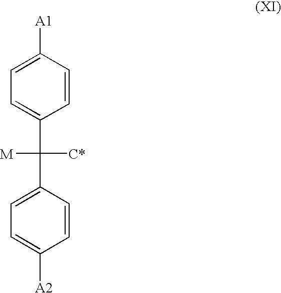
where M is a polycyclic aryl or heteroaryl group, A₁ and A₂ are in each case independently selected from H, O, OR, NHR or NR₂, wherein R is a C₁-C₂₀ hydrocarbon group which may optionally carry one or more heteroatoms, for example an alkyl, aryl, aralkyl or alkaryl group, and C* is a functional group, for example a linker synthon building block or a probe synthon building block. The group M is preferably an aryl or heteroaryl group having at least 3 or 4 fused rings, for example a pyrene group. M is furthermore preferably a fluorescent labeling group, for example a coumarin, a pyrene, Cy5, Cy3, a rhodamine or a fluorescent nanoparticle. M may, optionally, be connected with the backbone via a spacer.

Preference is given to at least one of the radicals A₁ and A₂ being NHR or NR₂. Particular preference is given to at least one of A₁ and A₂ being a dialkylamino group, the alkyl radicals having from 1 to 20 carbon atoms. Compounds (XI) in which A₁ or/and A₂ is an NHR or NR₂ group surprisingly exhibit a higher hydrolysis lability and thus better removability and higher color intensity.

The invention still further relates to compounds of the general structure (XII):

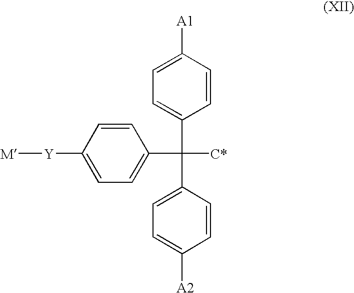

where M′ is a labeling group, Y is a bond or a spacer with a chain length of up to 20 carbon atoms and, optionally, one or more heteroatoms, A₁ and A₂ are in each case independently selected from H, O, OR, NHR or NR₂, wherein R is a C₁-C₂₀ hydrocarbon group which may optionally carry one or more heteroatoms, for example an alkyl, aryl, aralkyl or alkaryl group, and C* is a functional group, for example a linker synthon building block or a probe synthon building block. M′ is preferably a fluorescent labeling group, for example a coumarin group . . . .

At least one of the radicals A₁ and A₂ is NHR or NR₂, particularly preferably A₁ or/and A₂ is a dialkylamino group, with an alkyl radical being able to comprise up to 20 carbon atoms.

In the compounds (XII), Y is preferably a nonphotolabile structure, i.e. the group M′ cannot be removed from the backbone of the compound by illumination.

The compounds (XI) and (XII) are preferred examples of synthon building blocks for use in a process as described above.

### EXEMPLARY EMBODIMENTS

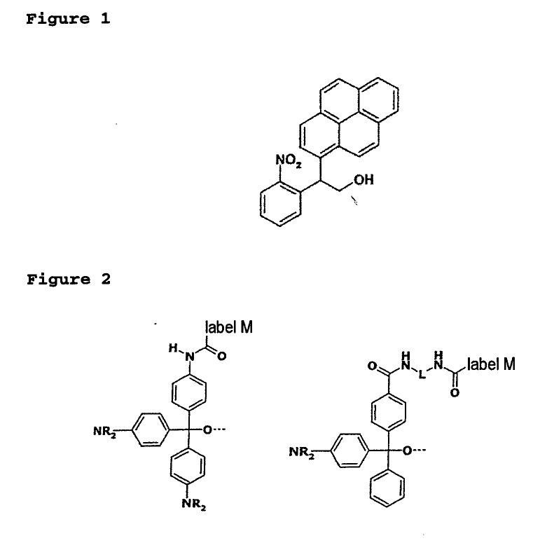

FIG. 1 depicts an exemplary embodiment of a photolabile protective group which may be used, for example, as group P^{m} in compounds of type I. Said protective group is an intramolecularly triplet-sensitized o-nitrophenylethyl photoprotective group which carries a pyrene radical. Further examples of suitable groups of this type are described in DE 102 60 592.0.

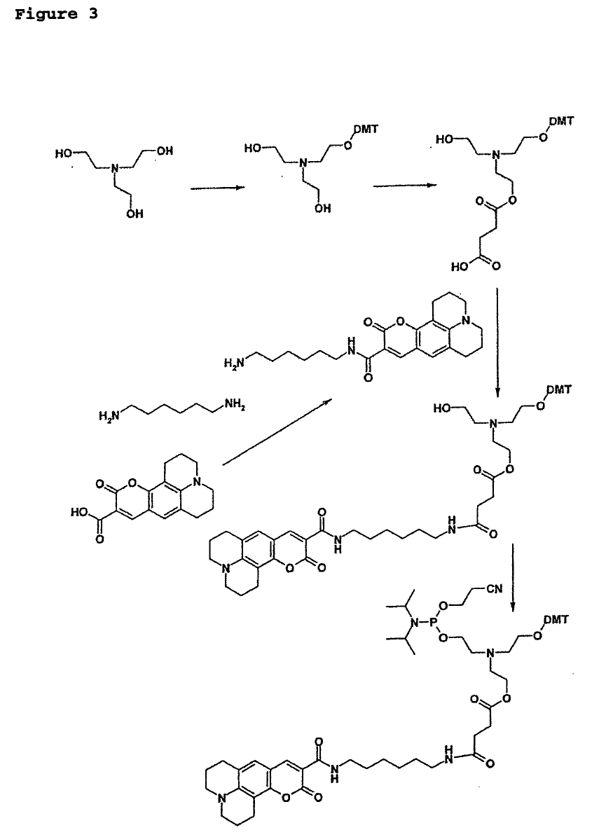

FIG. 2 depicts compounds of the type M-L-P^{m}-C* which may be used, for example, for compounds of the type II. P^{m} is an optionally substituted trityl group to which a label M is coupled, optionally, via a linker.

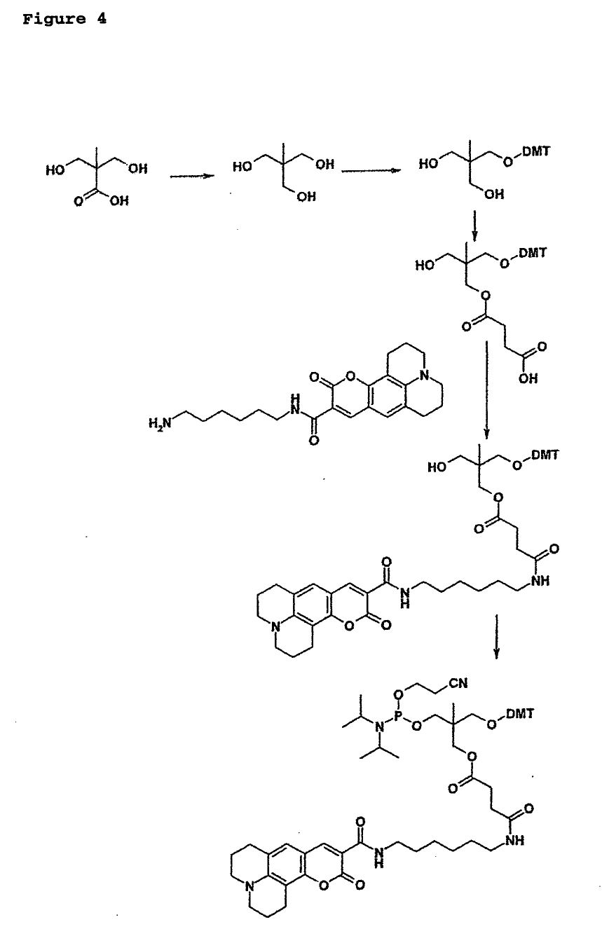

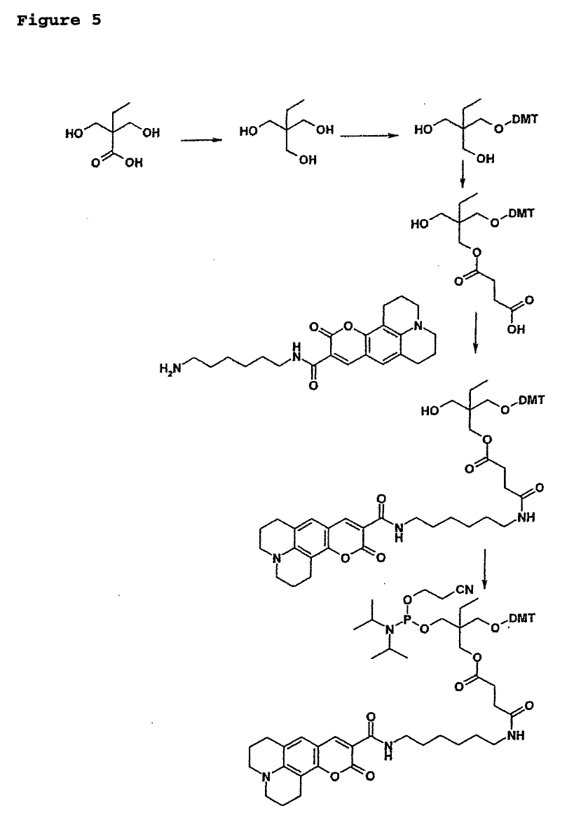

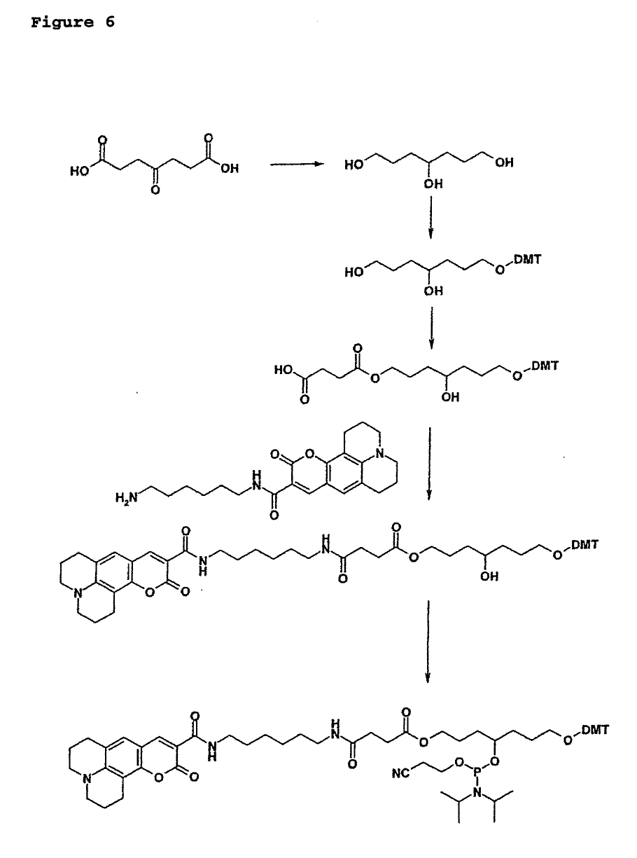

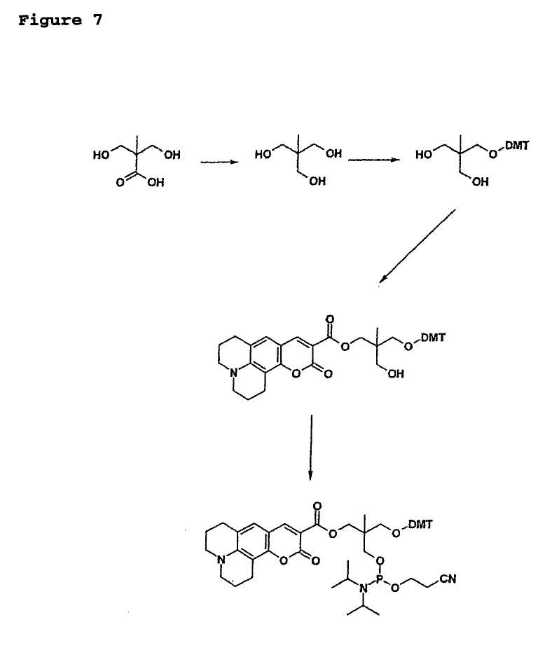

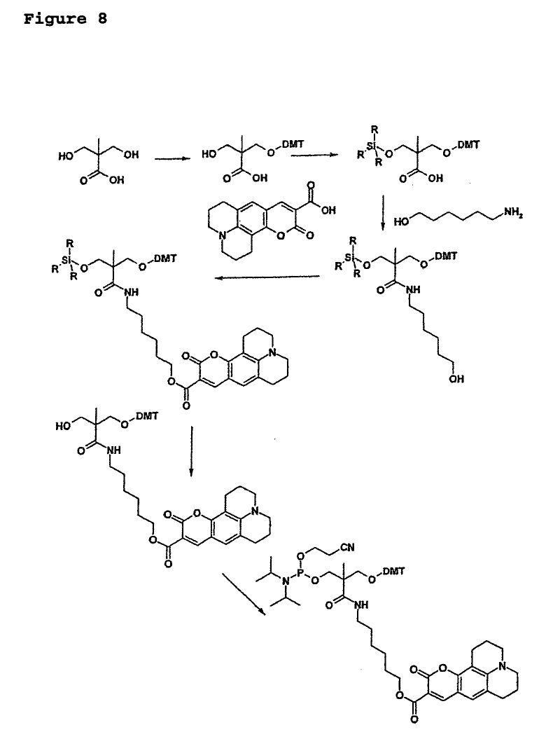

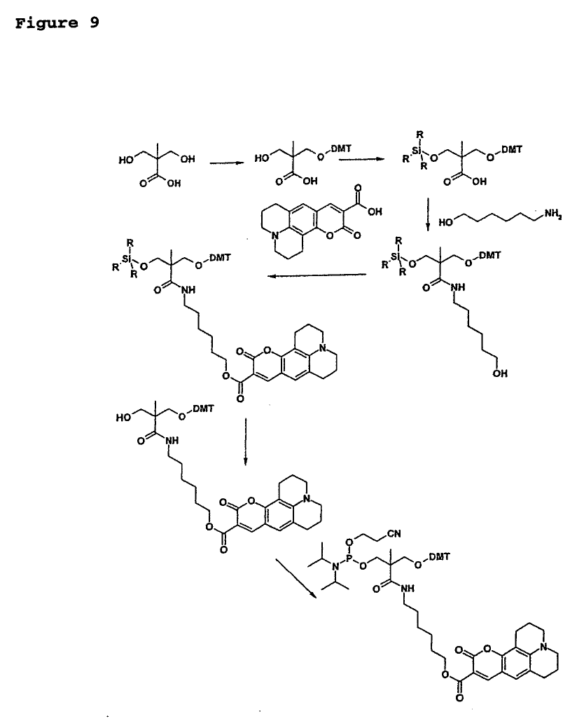

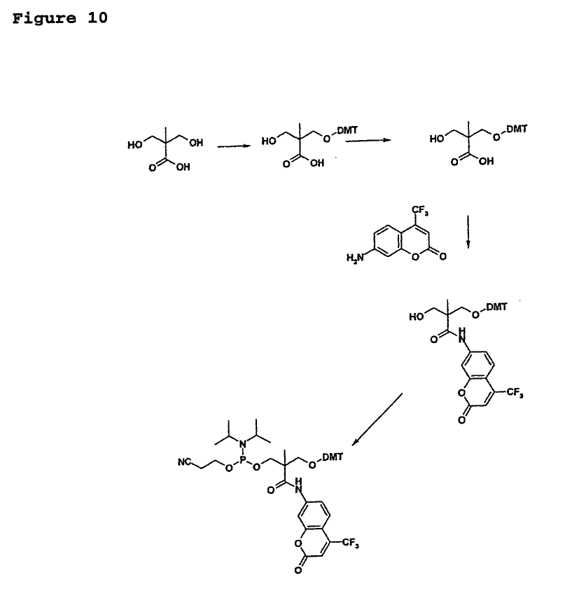

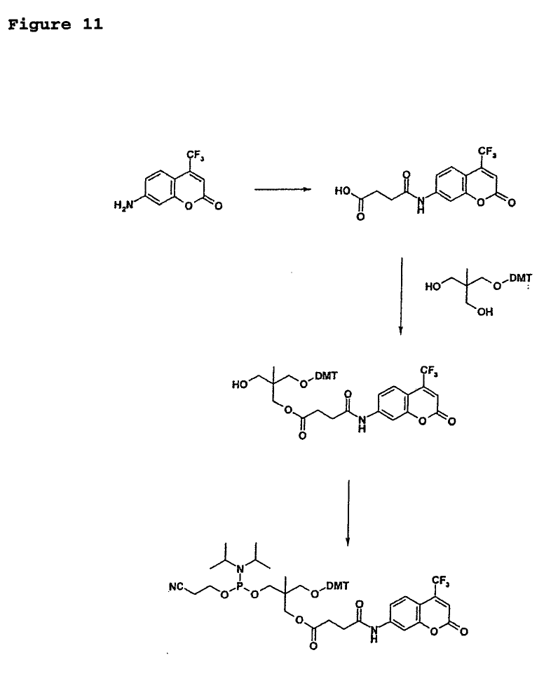

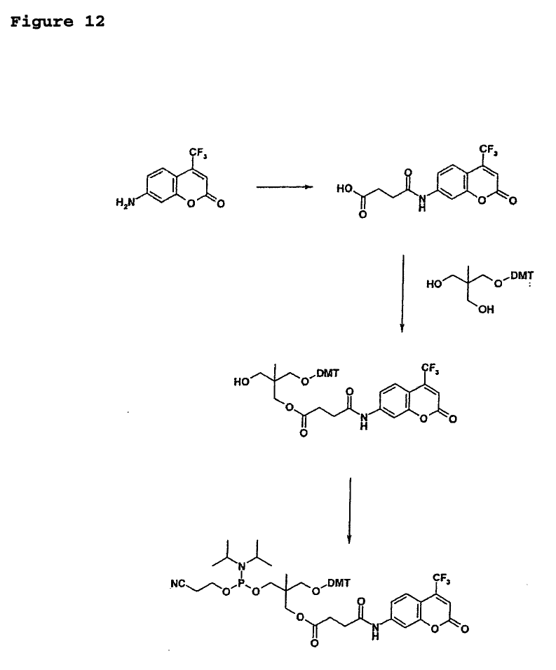

FIGS. 3 to 12 depict examples of compounds of type III (P^{m}-L-V (L′-H-L″-M)-L″′-C*) and the synthesis thereof. Said compounds are trifunctional molecules which provide one functionality for polymer synthesis, one for a label M and one for anchoring on the solid phase. The second functionality between label and trifunctional molecule has a labile bond. The following protective groups or protective group types are suitable for P^{m}: DMT protective group, Nppoc protective group, a two-stage protective group, a fluorescent two-stage protective group or a fluorescently labeled two-stage protective group.

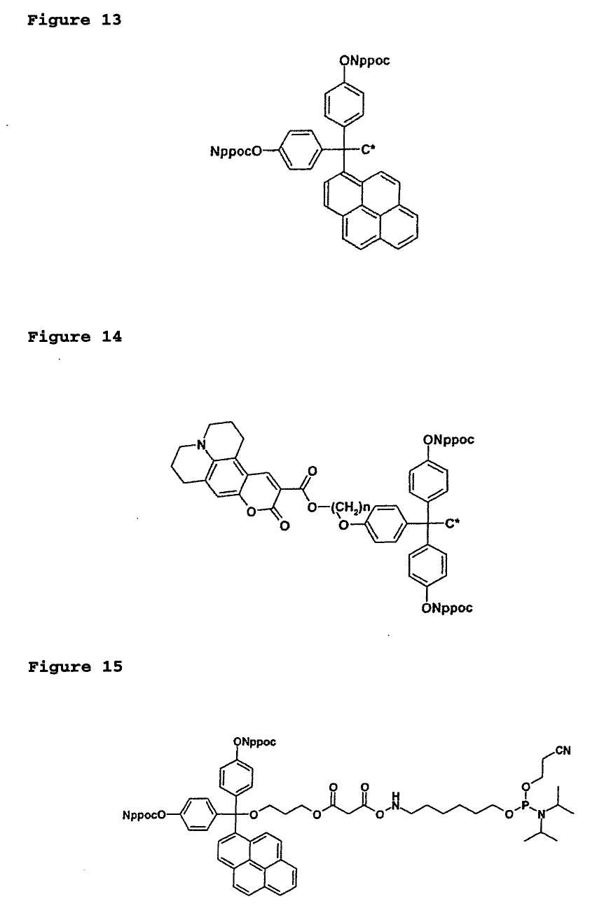

FIG. 13 depicts a compound of type VI (P^{m}′P^{m}-C*) which is a fluorescent two-stage protective group.

FIG. 14 is a compound of type VII (M-L-P^{m}′P^{m}-C*) which is likewise a fluorescently labeled two-stage protective group. P^{m} is the trityl group which is coupled via a linker to a coumarin group (M). Furthermore, photoactivatable NppoC groups (P^{m}′) are present on the trityl group.

FIG. 15 depicts a compound of type IX (P^{m}′P^{m}-L-H-L′-C*). This is a fluorescent two-stage protective group and a spacer which is interrupted by a predetermined breaking point H(OCO—CH₂—CH₂—CO—O).

1. A method for preparing a coated support, comprising the following steps:
providing a support whose surface has reactive groups,
constructing a functionalized surface on said support by synthesizing step-by-step a receptor comprising a linker element and a probe element made of synthon building blocks,
characterized in that
the synthesis process is monitored (i) by introducing one or more synthon building blocks with at least one detectable labeling group into the linker element and (ii) by introducing one or more synthon building blocks with at least one detectable labeling group into the probe element of the receptor.

2. The method as claimed in claim 1,
characterized in that synthon building blocks comprising a labeling group are used.

3. The method as claimed in claim 1,
characterized in that synthon building blocks comprising a plurality of labeling groups detectable independently are used.

4. The method as claimed in claim 1,
characterized in that labeling groups for introduction into the linker element are detectable beside labeling groups for introduction into the probe element.

5. The method as claimed in claim 1,
characterized in that one, two, three or all synthon building blocks for synthesizing the linker element comprise at least one detectable labeling group.

6. The method as claimed in claim 1,
characterized in that one, two, three or all synthon building blocks for synthesizing the probe element comprise at least one detectable labeling group.

7. The method as claimed in claim 1, characterized in that the synthesis process is monitored online.

8. The method as claimed in claim 1, characterized in that the probe elements of the receptor are selected from nucleic acids (in any directions of synthesis), such as DNA or RNA, nucleic acid analogs such as PNA or LNA, carbohydrates, peptides, derivatives of combinatorial chemistry and combinations thereof.

9. The method as claimed in claim 1, characterized in that monitoring comprises an optical measurement.

10. The method as claimed in claim 9, characterized in that the optical measurement comprises a determination of absorption, emission, light diffraction, light scattering or ellipsometry.

11. The method as claimed in, claim 1 characterized in that monitoring comprises a radioactivity measurement.

12. The method as claimed in claim 1, characterized in that monitoring comprises a plasmon resonance measurement.

13. The method as claimed in claim 1, characterized in that monitoring comprises an electronic measurement.

14. The method as claimed in claim 13, characterized in that the electronic measurement comprises a determination of electron diffraction or electrical signals.

15. The method as claimed in, claim 1 characterized in that the construction of the support is carried out in an integrated synthesis/analysis device.

16. The method as claimed in, claim 1 characterized in that at least one trifunctional synthon building block is used for constructing the linker or/and the probe.

17. The method as claimed in claim 1, characterized in that at least one synthon building block for incorporation into the linker element selected from any of the compounds (types I to VII) is used:

| | |
|---|---|
| Type I: | P^{m}-C* |
| Type II: | M-L-P^{m}-C* |
| Type III: | P^{m}-L-V(L′-H-L″-M)-L″′-C* |
| Type IV: | P^{m}′P^{m}-L-V(L′-H-L″-M)-L″′-C* |
| Type V: | M-L-P^{m}′P^{m}-L-V(L′- H-L″-M)-L″′-C* |
| Type VI: | P^{m}′P^{m}-C* |
| Type VII: | M-L-P^{m}′P^{m}-C* |

where
P^{m} is a protective group P which may carry a label m for detection, said protective group and said label being compatible with synthesis chemistry,
P^{m}′ is a protective group P orthogonal to P^{m}, which may carry a label m′ for detection, wherein
M is a label which may be used for detection, wherein m, M and, optionally, m′ are detectable independently and wherein the labels are compatible with synthesis chemistry,
L-L″ are any spacers, for example organic groups,
H is a cleavable group,
V is a trifunctional molecule/atom and
C* is a functional group or a functionalized support surface.

18. The method as claimed in claim 17, characterized in that at least one of the compounds of types (I) or/and (II) is used.

19. The method as claimed in claim 17, characterized in that C* is a linker building block.

20. The method as claimed in claim 1, characterized in that at least one synthon building block for incorporation into the probe element selected from any of the compounds of types (I, II or VI to X) is used:

| | |
|---|---|
| Type I: | P^{m}-C* |
| Type II: | M-L-P^{m}-C* |
| Type VI: | P^{m}′P^{m}-C* |
| Type VII: | M-L-P^{m}′P^{m}-C* |
| Type VIII: | P^{m}-L-H-L′-C* |
| Type IX: | P^{m}′P^{m}-L-H-L′C* |
| Type X: | M-L-P^{m}′P^{m}-L-H-L″-C * |

where
P^{m} is a protective group P which may carry a label m for detection, said protective group and said label being compatible with synthesis chemistry,
P^{m}′ is a protective group P orthogonal to P^{m}, which may carry a label m′ for detection, wherein said protective group and said label are compatible with synthesis chemistry,
M is a label which may be used for detection, wherein m, M and, optionally, m′ are detectable independently and wherein the labels are compatible with synthesis chemistry,
L-L″ are any spacers,
H is a cleavable group and
C* is a probe building block.

21. The method as claimed in claim 20, characterized in that at least one of the compounds of types (I) or/and (II) is used.

22. A compound of the general structure (XI): 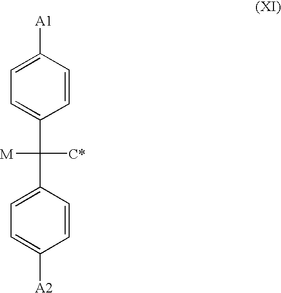
where
M is a polycyclic aryl or heteroaryl group,
A₁ and A₂ are in each case independently selected from H, O, OR, NHR or NR₂, wherein R is a C₁-C₂₀ hydrocarbon group which may optionally carry one or more heteroatoms, for example an alkyl, aryl, aralkyl or alkaryl group, and
C* is a functional group.

23. The compound as claimed in claim 22, characterized in that M is an aryl or heteroaryl group having at least 3 or 4 fused rings.

24. The compound as claimed in claim 23, characterized in that M is a pyrene group.

25. The compound as claimed in, claim 22 characterized in that at least one of A₁ and A₂ is NHR or NR₂.

26. A compound of the general structure (XII): 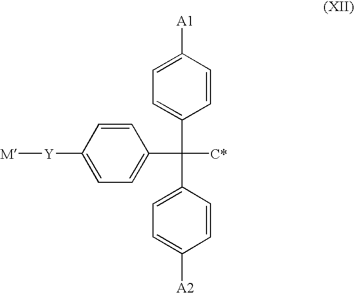
where
M′ is a labeling group,
Y is a bond or a spacer with a chain length of up to 20 carbon atoms and, optionally, one or more heteroatoms,
A₁ and A₂ are in each case independently selected from H, O, OR, NHR or NR₂, wherein R is a C₁-C₂₀ hydrocarbon group which may optionally carry one or more heteroatoms, for example an alkyl, aryl, aralkyl or alkaryl group, and
C* is a functional group.

27. The compound as claimed in claim 26, characterized in that M′ is a fluorescent labeling group.

28. The compound as claimed in claim 26, characterized in that M′ is coumarin, fluorescein, pyrene, Cy5, Cy3, rhodamine or a nanoparticle.

29. The compound as claimed in, claim 26 characterized in that at least one of A₁ and A₂ is NHR or NR₂.

30. The compound as claimed in, claim 26 characterized in that Y comprises a nonphotolabile structure.

31. The use of a compounds of the general structure (XI): 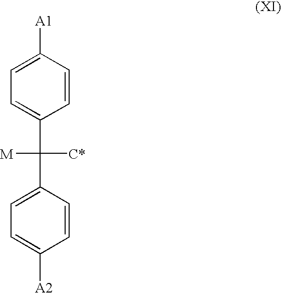
where
M is a Polycyclic aryl or heteroaryl group,
A₁ and A₂ are in each case independently selected from H, O, OR, NHR or NR₂, wherein R is a C₁-C₂₀ hydrocarbon group which may optionally carry one or more heteroatoms, for example an alkyl, aryl, aralkyl or alkaryl group, and
C* is a functional group.
as a synthon building blocks in a method as claimed in claim 1.
